(19) United States
(12) Patent Application Publication
(10) Pub. No.: US 2023/0128372 A1
(43) Pub. Date: Apr. 27, 2023

(54) AZD1656 FOR USE IN THE TREATMENT OF PNEUMONITIS AND/OR MYCORDITIS CAUSED BY A CORONAVIRUS

(71) Applicant: EXCALIBUR MEDICINES LIMITED, St James's (GB)

(72) Inventor: John Francis MARTIN, London (GB)

(21) Appl. No.: 17/911,644

(22) PCT Filed: Mar. 12, 2021
(86) PCT No.: PCT/GB2021/050623

§ 371 Date: Sep. 14, 2022

(30) Foreign Application Priority Data
Mar. 14, 2020 (GB) 2003722.2

(51) Int. Cl. A61K 31/497; A61P 29/00; A61P 11/00; A61P 3/10

(52) U.S. Cl. CPC A61K 31/497; A61P 29/00; A61P 11/00; A61P 3/10

(57) ABSTRACT

The present invention relates to AZD1656 or a pharmaceutically acceptable salt thereof, for use in the treatment of pneumonitis and/or myocarditis, which may be associated with or caused by a coronavirus infection.

### FIELD OF THE INVENTION

This invention relates to the treatment of pneumonitis (destructive inflammation of the lungs) and/or myocarditis (destructive inflammation of the heart).

### BACKGROUND OF THE INVENTION

Pneumonitis is a general term that refers to inflammation of lung tissue. Common causes of pneumonitis include airborne irritants or a side-effect of certain drug treatments.

The most common symptom of pneumonitis is shortness of breath, which may be accompanied by a dry cough. If pneumonitis is undetected or left untreated, the patient may develop chronic pneumonitis, which can result in scarring (fibrosis) in the lungs.

Myocarditis is an inflammation of the heart muscle (myocardium). Myocarditis can affect the heart muscle and the heart rhythm, reducing the heart's ability to pump and causing rapid or abnormal heart rhythms (arrhythmias).

Pneumonitis and myocarditis may be associated with viral infection. One such viral infection is a coronavirus, such as Covid-19. In the present coronavirus pandemic, causes of death from the viral infection are pneumonitis and/or myocarditis and there exists a need for new drugs to treat these conditions.

Glucokinase is an enzyme that facilitates phosphorylation of glucose to glucose-6-phosphate. Glucokinase occurs in cells in the liver and pancreas of humans and most other vertebrates. In each of these organs it plays an important role in the regulation of carbohydrate metabolism by acting as a glucose sensor, triggering shifts in metabolism or cell function in response to rising or falling levels of glucose, such as occur after a meal or when fasting. Mutations of the gene for this enzyme can cause unusual forms of diabetes or hypoglycemia. Glucokinase (GK) is a hexokinase isozyme, related homologously to at least three other hexokinases.

AZD1656 is a potent, selective (>100-fold versus hexokinase 1 and 2 and a pharmacology screening panel), activator of human and rat glucokinase in vitro; EC₅₀'s=0.057 and 0.072 μM, respectively, for the recombinant enzymes, which translates into cellular systems (EC50's=1.39 and 0.47 μM in human and rat hepatocytes, respectively). AZD1656 reduces plasma glucose levels in a dose-dependent fashion, with a rapid onset of action, in normo-glycaemic insulin resistant rats and diabetic mice, when dosed acutely and when dosed once daily for up to 28 days.

AZD1656 has been studied in single doses of up to 180 mg and multiple doses to 150 mg BID for 8 days in healthy volunteers as well as alone and in combination with other blood glucose control agents in diabetic patients at 200 mg daily for up to 6 months duration. In both healthy volunteers and diabetic patients no significant clinical effects other than glucose lowering were noted.

Preclinical studies of up to 12-month duration have been performed. These revealed a potent glucose lowering effect, and thereby, the results of chronic toxicology studies in healthy animals were confounded by severe hypoglycaemia at higher doses and sequalae such as Wallerian type nerve degeneration and skeletal muscle fibre degeneration. Additional changes, also considered secondary to hypoglycaemia, were seen in the liver (loss of hepatocellular glycogen).

In a Phase 2 study in Japanese type 2 diabetic subjects, AZD1656, given BID at high (40-200 mg/day), medium (20-140 mg/day) and low (10-80 mg/day) doses over a 4 month period, has been found to lower HbA1c and fasting plasma blood (FPG) glucose levels with a 50 mg dose producing compound levels of ˜2×EC₅₀ in plasma. However, this effect was transient trending towards pre-dose levels between weeks 8 and 16 and there was no statistically significant change in either HbA1c or FPG from baseline at 4 months.

AZD1656 has been shown not to cause hypoglycaemia in healthy, non-diabetic patients when administered in doses of 40 mg or 80 mg (hypoglycaemia was defined in the study as 2.7 mmol/l) (Norjavaara E. et al., J Clin Endocrinol Metab, 2012, 97(9):3319-3325).

AZD1656 has the systematic name 3-{[5-(Azetidin-1-ylcarbonyl)pyrazin-2-yl]oxy}-5-{[(1S)-1-methyl-2-(methyloxy) ethyl]oxy}-N-(5-methylpyrazin-2-yl)benzamide.

AZD1656 has the structure:

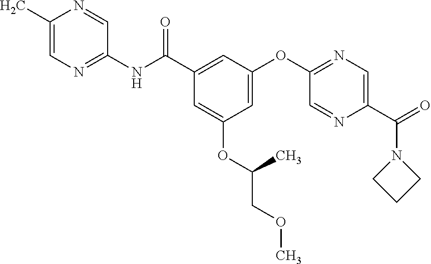 [SMILES: COC[C@H](C)Oc1cc(Oc2cnc(C(=O)N3CCC3)cn2)cc(C(=O)Nc2cnc(C)cn2)c1]

### SUMMARY OF THE INVENTION

The present invention is AZD1656, or a pharmaceutically acceptable salt thereof, for use in the treatment of pneumonitis and/or myocarditis, particularly as a result of a coronavirus infection.

AZD1656 is a molecule which is an activator of glucokinase in regulatory T cells (Tregs). These cells are a specialised sub-population of T cells that act to suppress the immune response. In inappropriate inflammation, Tregs can suppress T cell proliferation and cytokine production. This may be clinically beneficial.

It is therefore predicted that administration of AZD1656, in particular when given either orally, transdermally or intravenously will be efficacious in the therapy of pneumonitis and/or myocarditis, in particular pneumonitis and/or myocarditis associated with a coronavirus infection, such as Covid-19.

Accordingly, a first aspect of the invention is AZD1656, or a pharmaceutically acceptable salt thereof, for use in the treatment of pneumonitis and/or myocarditis.

A second aspect of the invention is a use of AZD1656, or a pharmaceutically acceptable salt thereof, for the manufacture of a medicament for use in the treatment of pneumonitis and/or myocarditis.

A third aspect of the invention provides a method of treating pneumonitis and/or myocarditis comprising administering a patient with AZD1656 or a pharmaceutically acceptable salt thereof.

### DETAILED DESCRIPTION

By the term “treatment” or “treating” as used herein, we refer to therapeutic (curative) treatment including amelioration. Treatment also includes stopping the disease from developing or slowing further progression of the disease. For example, treatment may include preventing symptoms from worsening. “Amelioration” is an improvement, or perceived improvement, in the patient's condition, or a change in a patient's condition that makes it, or side-effects, increasingly tolerable.

Pneumonitis and/or myocarditis are often caused by a viral infection. Thus, in one embodiment, pneumonitis and/or myocarditis is characterised as being as a result of or associated with a viral infection, preferably a coronavirus infection, for example SARS (severe acute respiratory syndrome) or SARS-CoV-2, and preferably COVID-19 or long Covid.

In one embodiment, the subject to be treated is infected with, or suspected to be infected with, a coronavirus, such as Covid-19. In a further embodiment, the subject infected with, or suspected to be infected with, a coronavirus is categorised as stage 3, 4, or 5 on the WHO Ordinal Scale for Clinical Improvement. WHO Ordinal Scale for Clinical Improvement measures illness severity over time (Michael O'Kelly & Siying Li (2020), Statistics in Biopharmaceutical Research, 12:4, 451-460).

“Covid-19” refers to an infectious disease caused by the virus severe acute respiratory syndrome coronavirus 2 (SARS-CoV-2).

“Long Covid” or “Post-Covid Syndrome” refers to signs and symptoms that develop during or following an infection consistent with COVID-19 which continue for more than 12 weeks and are not explained by an alternative diagnosis. The condition usually presents with clusters of symptoms, often overlapping, which may change over time and can affect any system within the body. Many people with post-COVID syndrome can also experience generalised pain, fatigue, persisting high temperature and psychiatric problems.

“Patient” and “subject” are used interchangeably and refer to the subject that is to be administered the AZD1656. Preferably, the subject is a human.

The invention is suitable for use in both diabetic and non-diabetic patients. In some embodiments, the subject has diabetes, preferably type 1 or type 2 diabetes.

In some embodiments, the subject has a blood glucose level at or above 4 mmol/L.

In some embodiments, the subject has diabetes and/or has undergone renal transplant.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

The skilled practitioner would understand that the compound of the invention may be formulated as a pharmaceutical composition comprising AZD1656 or a pharmaceutically acceptable salt thereof. In one embodiment, AZD1656 is the only active agent in the composition. By only active agent it is meant that the composition does not contain other components which may be used in the treatment of pneumonitis and/or myocarditis, and/or a viral infection.

The compositions comprising AZD1656, or a pharmaceutically acceptable salt thereof, may contain a pharmaceutically acceptable carrier. By “pharmaceutically acceptable carrier” is meant any diluent or excipient, such as fillers or binders, that is compatible with the other ingredients of the composition, and which is not deleterious to the recipient. The pharmaceutically acceptable carrier can be selected on the basis of the desired route of administration, in accordance with standard pharmaceutical practices.

In the present invention, AZD1656 may be administered in a variety of dosage forms. In one embodiment, AZD1656 may be formulated in a format suitable for oral, rectal, parenteral, intranasal or transdermal administration or administration by inhalation or by suppository.

AZD1656 may be administered orally, for example as tablets, troches, lozenges, aqueous or oily suspensions, dispersible powders or granules. Preferably, AZD1656 is formulated such that it is suitable for oral administration, for example tablets and capsules.

AZD1656 may also be administered parenterally, whether subcutaneously, intravenously, intramuscularly, intrasternally, transdermally or by infusion techniques. AZD1656 may also be administered as suppositories.

AZD1656 may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed.

The present invention also provides an inhalation device containing AZD1656. Typically said device is a metered dose inhaler (MDI), which contains a pharmaceutically acceptable chemical propellant to push the medication out of the inhaler.

AZD1656 may also be administered by intranasal administration. The nasal cavity's highly permeable tissue is very receptive to medication and absorbs it quickly and efficiently. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing AZD1656.

AZD1656 may also be administered by transdermal administration. For topical delivery, transdermal and transmucosal patches, creams, ointments, jellies, solutions or suspensions may be employed. The present invention therefore also provides a transdermal patch containing a AZD1656.

AZD1656 may also be administered by sublingual administration. The present invention therefore also provides a sub-lingual tablet comprising AZD1656.

AZD1656 may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

In an embodiment of the invention, AZD1656 is administered in an effective amount to treat the symptoms of pneumonitis and/or myocarditis. An effective dose will be apparent to one skilled in the art and is dependent on a number of factors including age, sex, weight, which the medical practitioner will be capable of determining.

In a preferred embodiment, AZD1656 is administered in doses of 0.5 to 400 mg, more preferably 1 to 400 mg, more preferably 2.5 to 400 mg, more preferably 5 mg to 400 mg, more preferably 50 mg to 300 mg, most preferably 150 mg to 300 mg. The lower limit for a dose is preferably 0.5 mg, 1 mg, 1.5 mg, 2 mg, 2.5 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg or 200 mg. The upper limit for a dose is preferably 400 mg, 390 mg, 380 mg, 370 mg, 360 mg, 350 mg, 340 mg, 330, mg, 320 mg, 310 mg, 300 mg, 290 mg, 280 mg, 270 mg, 260 mg, 250 mg, 240 mg, 230 mg, 220 mg or 210 mg. Any of the aforementioned lower or upper limits of the ranges may be combined with each other and are herein disclosed. In some embodiments, the dose is 150 mg to 300 mg. In some embodiments, the dose is 2 to 100 mg or about 2.5 mg.

Any of the above doses may be administered once a day, twice a day, three times a day or four times a day.

In an embodiment of the invention, AZD1656 is administered at least once a day. Preferably it is administered as a single daily dose. Preferably the single daily dose is of 200 mg to 400 mg or 2 to 100 mg. Preferably it is 2.5 mg, 200 mg, 300 mg or 400 mg.

It will be appreciated that a lower dose may be needed in a paediatric patient.

In an embodiment of the invention, AZD1656 is administered twice daily. Preferably each dose is 1 to 20 mg or 150 mg to 200 mg, with a total daily dosage of 2 to 40 mg or 300 mg to 400 mg.

Alternatively, it may be administered three times per day. Preferably each dose is 1 to 20 mg or 100 mg to 130 mg.

Alternatively, it may be administered four times per day. Preferably each dose is 1 to 20 mg or 75 mg to 100 mg.

Preferably, the dosage regime is such that the total daily dosage of AZD1656 does not exceed 400 mg, more preferably 300 mg.

In order to treat pneumonitis and/or myocarditis, AZD1656 is used in a chronic dosage regime i.e. chronic, long-term treatment.

The present invention also relates to use of AZD1656, or a pharmaceutically acceptable salt thereof, for the manufacture of a medicament for use in the treatment of pneumonitis and/or myocarditis. This embodiment of the invention may have any of the preferred features described above.

In one embodiment, the pneumonitis and/or myocarditis is associated with a coronavirus infection, for example Covid-19 or long Covid.

The present invention also relates to a method of treating pneumonitis and/or myocarditis comprising administering the patient with AZD1656 or a pharmaceutically acceptable salt thereof. This embodiment of the invention may have any of the preferred features described above. The method of administration may be according to any of the routes described above.

For the avoidance of doubt, the present invention also embraces prodrugs which react in vivo to give a compound of the present invention.

The following examples illustrate the invention.

### EXAMPLES

Study

The first clinical trial is in patients with diabetes with proven coronavirus infection. These patients are at an increased risk of death compared to non-diabetics. The endpoints are lung imaging, inflammatory markers in the blood, arterial oxygen concentration and cardiac ejection fraction and survival. Later trials of the compound will be undertaken in diabetic and non-diabetic patients when given on the first presentation with symptoms. The compound should prevent and treat lung and heart disease in coronavirus infection.

The first (Phase II) clinical trial is currently being conducted in the UK, Romania and Czech Republic to assess the safety and efficacy of AZD1656 hospitalised with suspected or confirmed COVID-19. At the time of filing this application, the data from the trial have yet to be unblinded. However, preliminary observations are available. As of Mar. 9, 2021, 116 patients have been entered into the trial and there are 5 deaths reported. At the independent Safety Review Committee (SRC) meeting on Feb. 26, 2021 the data from 60 patients having completed the trial so far were examined from the point of view of safety without breaking the blinding code. The quality of the data was deemed high. Of the 60 patients who at that time had completed the trial protocol 5 had died. The Committee had no safety concerns. Patient entry characteristics were equally distributed between test and control group.

The French Coronad study of diabetic patients with Covid-19 reported that the death rate of diabetic patients in hospital because of Covid-19 was 1 in 5 within 28 days. Thus, in line with the Coronad study data, of the 60 patients who had completed the current trial by February 26^{th}, one would expect 12 deaths. If the number of deaths was randomly distributed between the test and control arms of the trial one would expect 6 deaths in the test group and 6 deaths in the control group according to the data from the Coronad study. However, by February 26^{th} there were 5 deaths overall (and not 12) in test and control group combined. The control group received an inactive placebo so it is reasonable to propose that the death rate in the placebo group would be 6 based on the Coronad study data. The test group received a dose of AZD1656 known to activate Treg lymphocytes which damp down inappropriate inflammation. The credibility of this hypothesis in Covid-19 disease was supported by the UK Government UKRI which is funding the trial and by the UK regulator the MHRA. It is therefore beyond reasonable doubt that the decrease of the expected death rate from an expected 12 to 5 people in the study up to February 26^{th} occurred in the AZD1656 group. It is submitted that these findings support the efficacy of AZD1656 in human patients with diabetes suffering from Covid-19 disease.

Study Design

This is a Phase II, randomised, placebo-controlled, double-blind clinical trial to assess the safety and efficacy of AZD1656 on the cardiorespiratory complications of COVID-19 in hospitalised diabetic patients with known or suspected COVID-19.

All patients will receive usual care plus either AZD1656 or placebo. The World Health Organization (WHO) 8-point Ordinal Scale for Clinical Improvement is used as the standard methodology for measuring patient outcomes.

Primary endpoints:

The primary endpoint is clinical improvement measured as the percentage of subjects at Day 14 who are in categories 1-3 according to the WHO 8-point Ordinal Scale for Clinical Improvement, comparing AZD1656 treatment to placebo.

Secondary endpoints:

(a) Clinical improvement measured as the percentage of patients categorised at each severity rating on the WHO 8-point Ordinal Scale at Day 7, Day 14 and Day 21 versus baseline, comparing AZD1656 treatment with placebo.

(b) Degree of glycaemic control as measured by the need to increase baseline medication requirements or the need to add additional diabetic medications to maintain appropriate blood glucose levels in patients receiving AZD1656 compared with placebo.

(c) Proportion of Treatment Emergent Adverse Events (TEAEs) leading to study drug discontinuation in patients receiving AZD1656 compared with placebo.

(d) Proportion of Serious Adverse Events (SAEs) in patients receiving AZD1656 compared with placebo.

(e) Time from hospital admission to hospital discharge (in hours) in patients receiving AZD1656 compared with placebo.

(f) Time from hospital admission to receiving intubation/mechanical ventilation in patients receiving AZD1656 compared with placebo.

(g) Mortality Rate in patients receiving AZD1656 compared with placebo.

Exploratory Endpoints:

(a) Plasma AZD1656 levels during first 7 days of treatment in patients receiving AZD1656 compared with placebo.

(b) Immunophenotyping panel to be conducted by Flow Cytometry: between group comparison (AZD1656 versus placebo) of levels of T, B and NK cells (including specific Treg and memory T cell populations); monocyte, neutrophil and eosinophil numeration to include activation markers for neutrophils (CD11b) and Monocytes subsets (CD14/CD16 identification including 6-Sulfo LacNAc(SLAN)).

(c) Immunochemistry panel to be conducted using the MSD U-Plex multiplex assay for assessment of the following biomarkers: G-CSF, GM-CSF, IL-1B, IL-4, IL-6, IL-8, IL-10, IL-12, and MIP-1a.

(d) Measurement of hsTroponin and NTproBNP to determine extent of cardiac injury in patients receiving AZD1656 compared with placebo.

(e) Measurement of 25-hydroxyvitamin D levels before treatment to determine whether there is any correlation between Vitamin D level and clinical outcomes.

(f) Correlation of clinical outcomes with patient ethnicity.

Study Protocol

Approximately 165 patients have been screened to achieve 150 patients randomly assigned to AZD1656 or placebo. All patients receive usual care plus either AZD1656 or placebo.

Inclusion Criteria

1. Male or Female.

2. Aged 18 and older.

3. Have either type 1 or type 2 diabetes.

4. Hospitalised with suspected or confirmed novel coronavirus (Severe Acute Respiratory Syndrome Coronavirus 2 (SARS-CoV-2)) infection at time of enrolment, categorised as stage 3, 4 or 5 on the WHO Ordinal Scale for Clinical Improvement.

5. Blood glucose level at or above 4 mmol/L.

6. Able to take oral (tablet) formulation of medication.

7. Patient is able to provide written informed consent prior to initiation of any study procedures.

Dose

AZD1656 administered orally, as 50 mg film-coated tablets to be dosed at 100 mg

BID (total daily dose of 200 mg).

The tablets were manufactured by Patheon.

Duration

A treatment duration of 21 days has been selected to explore the effects of AZD1656 on safety and efficacy. Treatment durations of up to 6 months have been evaluated in previous trials, so the duration in this trial is considered safe and is appropriate for this patient population based on the expected duration of hospitalisation for diabetic patients requiring hospital treatment for COVID-19.

The study has 3 phases:

1. Screening/Randomisation (Day −2 to 1)

2. Double-Blind treatment (Day 1 to Day 21)

3. Safety Follow-up (7 days after completion of the study treatment)

The study evaluates efficacy across a range of assessments evaluating clinical improvement, glycaemic control, time from hospital admission to hospital discharge and time from hospital admission to receiving intubation/mechanical ventilation.

1. A method of treating pneumonitis and/or myocarditis, comprising administering 3-{[5-(azetidin-1-ylcarbonyl)pyrazin-2-yl] oxy}-5-{[(1 S)-1-methyl-2-(methyloxy) ethyl]oxy}-N-(5-methylpyrazin-2-yl)benzamide (AZD1656), or a pharmaceutically acceptable salt thereof to a subject in need thereof.

2. The method according to claim 1, wherein the subject of treatment has diabetes.

3. The method according to claim 2 wherein the subject of treatment is human.

4. The method according to claim 1, wherein AZD1656 is administered in a total daily dose of 1 to 400 mg, or 50 mg to 400 mg, or 50 mg to 300 mg, or 150 mg to 300 mg.

5. The method according to claim 1 wherein the administering is by a single daily dose.

6. The method according to claim 5, wherein the single daily dose is 1 to 400 mg, or 200 mg.

7. The method according to claim 1, where the administering administration is by a dose twice per day.

8. The method according to claim 7, wherein each of the two doses per day is 100 mg.

9. The method according to claim 1, wherein AZD1656 is administered in a dose of 40 to 300 mg.

10. The method according to claim 1, wherein the lower limit for a dose of AZD1656 is 80 mg and the higher limit for a dose of AZD1656 is 210 mg.

11. The method according to claim 1, wherein AZD1656 is administered orally.

12. The method according to claim 1, wherein AZD1656 is to be administered by parenteral, transdermal, sublingual, rectal or inhaled administration.

13. The method according to claim 1 wherein the pneumonitis and/or myocarditis is associated with or caused by a coronavirus infection, such as Covid-19.

14. The method according to claim 1 wherein the subject to be treated is infected with, or suspected to be infected with, a coronavirus.

15. The method according to claim 1 wherein the subject to be treated has long COVID.

16. (canceled)

17. (canceled)

18. (canceled)

19. (canceled)

20. The method according to claim 2, wherein the total daily dose of AZD1656 is 1 to 400 mg, or 50 mg to 400 mg, or 50 mg to 300 mg, or 150 mg to 300 mg.

21. The method according to claim 2, wherein the lower limit for a dose is 80 mg and the higher limit for a dose is 210 mg.

22. The method according to claim 2, wherein the administering is by a dose twice per day and each of the two doses per day is 100 mg.

23. The method according to claim 2, wherein the pneumonitis and/or myocarditis is associated with or caused by a coronavirus infection.

24. The method according to claim 2, wherein the diabetes is type 1 diabetes.

25. The method according to claim 2, wherein the diabetes is type 2 diabetes.
